(19) United States
(12) Patent Application Publication
(10) Pub. No.: US 2002/0119949 A1
(43) Pub. Date: Aug. 29, 2002

(54) Prophylactic teat treatment

(76) Inventors: Asa Hellman, Upplands Vasby (SE); Torbjorn Mathisen, Alvsjo (SE)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 09/791,739

(22) Filed: Feb. 26, 2001

(51) Int. Cl. A61K 31/721; A61K 31/722; A61K 31/727

(52) U.S. Cl. 514/55; 514/56; 514/59

(57) ABSTRACT

An aqueous solution for prophylactic treatment of teats of lactating mammals, comprising as a first component at least partially deacetylated chitosan, or an acid addition salt thereof, in a concentration of up to about 2% by weight of chitosan, the solution having a pH of from about 4 to about 6.8, and said first component having a molecular weight such that the viscosity of the solution is less than about 50 mPas; and

a method of prophylactic treatment of teats of lactating mammals.

### TECHNICAL AREA

The present invention relates to aqueous preparations for the prophylactic treatment of teats of lactating animals and includes a method for prophylactic treatment of such teats to prevent onset or outbreak of mastitis. Although the invention is involved with chemical substances known par se, the invention provides for new formulations resulting in improved properties and better results of treatment.

### BACKGROUND OF THE INVENTION

Mastitis is an inflammatory reaction of udder tissue and is the most common and most costly disease among lactating cows over the world. The inflammation is a reaction of the lactating tissues on the presence of infectious microorganisms. A large number of different bacteria have boon identified as mastitis pathogens. They have been divided into four different groups, contagious, environmental, opportunistic and other bacteria. The majority of the mastitis infections are caused by S. aureus. Another contagious mastitis pathogen is Streptococcus agalectiae. Among the environmental bacteria there are other streptococci and the coliform bacteria, such as Escherichia coli and Klebsiella pneumoniae.

A large number of different disinfectants (most frequently chlorohexidine or iodophors) are used for dipping the teats immediately after milking in order to prevent bacteria from penetrating into the teat canal and further to lactating tissues. These disinfectants have a killing effect in direct contact between disinfectant and bacterium. In spite of routine use of these agents a number of bacteria escaped the killing effect, i e the known agents are not sufficiently effective, which can be due to insufficient sufficient amount of active components and the fact that the agents do not reach sufficient contact with the infected sites. Another disadvantage with these disinfectants is that they have a dehydrating effect on the skin, resulting in skin damage with extended use causing increased bacterial invasion, It is also known that the effect of these agents fades out very quickly and that renewed contamination of the teats takes place shortly after the treatment. Numerous studies have shown that small wounds and skin tissues on the teats can act as reservoirs for many types of bacteria, and livestock with infected teat wounds often show higher mastitis frequencies than other livestock. Furthermore, iodine and chlorohexidin can result in taste changes of the milk, and relatively small quantities of iodine and chlorohexidin in milk can cause problems in the manufacture of dairy products.

The state of the art presents other alternative agents for the prophylactic treatment of the teats of lactating animals. Accordingly, it is known from JP 2648686 to use chitosan or a derivative thereof in aqueous solution for such prophylactic treatment, the agent being applied by spraying or dipping, Although this piece of prior art teaches the use of acid aqueous solutions it does not specify any pH-ranges or any specifications of the chitosan used to prepare aqueous solutions. Also belonging to the state of the art is WO98/48627 disclosing similar aqueous solutions of chitosan but further including heparin, heparan sulphate or dextran sulphate without, however, specifying the chitosan used more in detail. Furthermore, this specification is totally silent with regard to any pH-value of the solutions disclosed.

The disadvantages experienced with the prior art preparations for the prophylactic treatment of the teats of lactating animals are of a different nature. First, the prior art solutions containing chitosan, by itself or in combination with additional components, have inferior storage stability and are therefore less suited for commercial purposes, which necessarily involve storage for various periods of time.

Seconds without specification of the pH of the solutions used, the anti-bacterial activity can vary between wide limits and the treatment may therefore be less efficient. Furthermore, at too low a pH, skin damage can occur with regular use.

Third, the way the solutions are applied to the teats, usually by spraying or dipping, makes it important to provide for aqueous solutions of substantially constant viscosity lying within specific limits. Application by spraying necessarily involves the use of solutions of specific viscosity to meet the requirements of the spraying equipment. Furthermore, both methods of application, i e spraying and dipping, again require specific values of viscosity in order to result in a situation whereby the solution remains on the teats and forms a stable layer lasting for a sufficient period of time.

### SUMMARY OF THE INVENTION

Considering the drawbacks of the prior art solutions and systems for prophylactic treatment of the teats of lactating animals, the present invention has for a main object to provide new solutions for such treatment which have substantially improved storage stability.

Another object of the invention is to provide solutions for such prophylactic treatment which have a high anti-microbial activity.

Yet another object of the invention is to provide solutions for such prophylactic treatment which have physical properties making them suitable for application by spraying or dipping to form a long-lasting layer or film resulting in extended anti-microbial effect.

Another object of the invention is to provide a method of prophylactic treatment of teats of lactating mammals efficiently preventing onset or outbreak of mastitis.

A further object of the invention is to provide a solution giving a skin conditioning effect.

To these and other objects which will become apparent from the following disclosure, the present invention provides for an aqueous solution for prophylactic treatment of teats of lactating mammals, such solution comprising as a first component at least partially deacetylated chitosan, or an acid addition salt thereof, in a concentration of up to about 2% by weight of chitosan, the solution having a pH of from about 4 to about 6.8, and said first component having a molecular weight such that the viscosity of the solution is less than about 50 mPas.

It is preferred that such aqueous solution according to the invention further comprises a second component selected from heparin, heparan sulphate and dextran sulphate, the weight ratio between said first and second components being from about 10:1 to about 100:1. Such a combination between said first and second components is particularly advantageous, in that it provides for an even more stable film or layer upon application.

To obtain maximum anti-bacterial efficiency it is preferred that the chitosan has a degree of deacetylation of no less than about 50%, such as no less than about 80%, and particularly no less than about 95%. However, an upper limit of the deacetylation degree of about 95% is preferred, mainly clue to practical and economic aspects of the preparation of chitosan. Thus, a preferred solution according to the invention comprises chitosan having a deacetylation degree within the range from about 85% to about 95%.

The pH of the solution of the invention is suitably maintained by the use of a mineral acid or organic acid. It is preferred that the acid is a weak organic acid providing a certain buffering effect resulting in a less varyinig pH of the solution. Particularly preferred weak organic acids are acetic acid and glycolic acid.

A preferred embodiment of the invention resides in an aqueous solution wherein the first component is chitosan in base form at a concentration of from about 0.2 to about 1.5% by weight, the weight ratio between said first and second components is from about 40:1 to about 60:1, and the acid is either acetic acid or glycolic acid.

For optimising the efficiency of the treatment the viscosity of the solution is preferably less than about 25 mPas, and a suitable range is from about 5 to about 20 mPas.

The molecular weight and the molecular weight distribution of the chitosan used is of importance to the stability of the solution thereof. In this regard, it is preferred that the molecular weight is such that a weight fraction in the range from about 70 to about 85% of the chitosan content has a moloecular wecight of from about 20 to about 150 kD, the molecular weight distribution of said weight fraction being centred around the weight average molecular weight (\overline{Mw}). It is particularly preferred that about 80% of the chitosan used has a molecular weight within the range from about 20 to about 150 kD. Characterisation of the weight average molecular weight (\overline{Mw}) and the molecular weight distribution of chitosans can be performed by size-exclusion chromatography (SEC) coupled with multiangle laser light scattering (MALLS) detection, as is known in the art.

It is preferred for optimizing the anti-microbial efficiency of the solution that the pH thereof is in the range from about 4.5 to about 6.

It will be understood by the skilled practitioner that any conventional and accepted excipients, additives and adjuvants may additionally be used in the solution according to the invention.

The invention also provides for a method of prophylactic treatment of teats of lactating animals to prevent onset or outbreak of mastitis, and this method resides in applying an aqueous solution as described above to said teats in an anti-microbially active amount.

Such application of the solution according to the invention suitably takes place in connection with milking operations. Considering the daily milking, the application is suitably performed twice or thrice a day after milking. In certain cases it may be preferable to apply the solution before milking, considering the risk of transferring micro-organisms during the milking process. The solution of the invention is very well suited for such a pre-milking treatment, as the risk of transferring non-desirable components into the milk is minimized.

It is preferred that the application of said solution is performed by dipping or spraying so as to cover at least the tip of the teats.

The solution according to the present invention can be prepared by dissolving the components thereof in water to give the desired concentration. An advantageous method in connection with the preparation of such solution resides in dissolving chitosan and other components separately in a fraction of the aqueous liquid used, for example about half of the quantity of the final solution.

The present invention will in the following be further described more in detail with reference to the appended drawings, wherein:

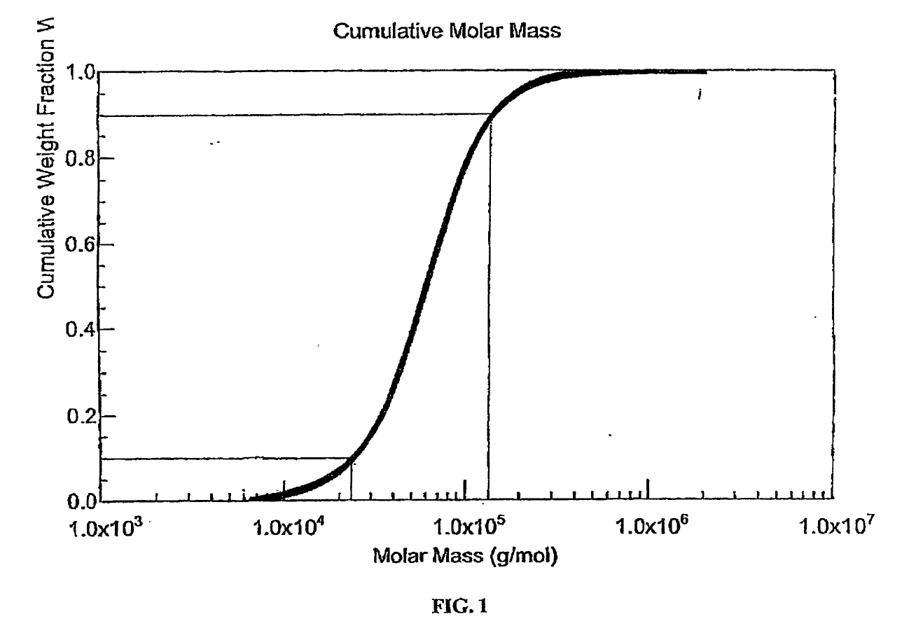

FIG. 1 is a diagram showing cumulative weight fraction versus molar mass;

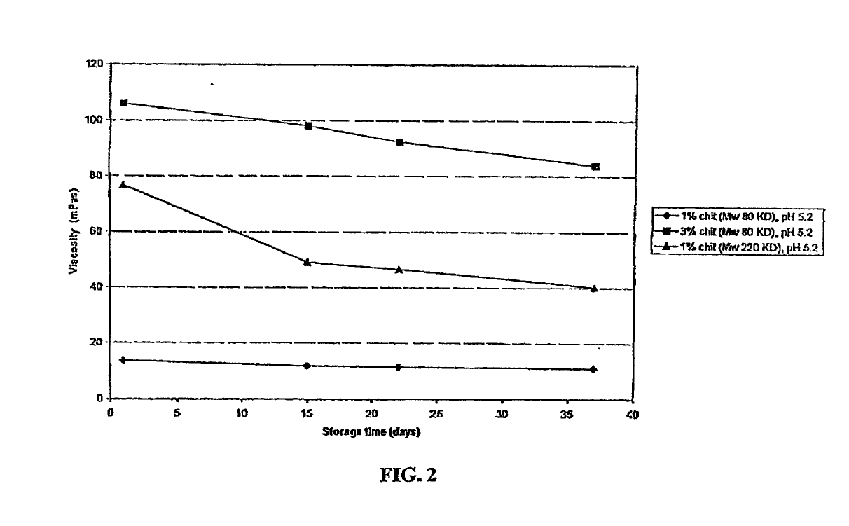

FIG. 2 shows a diagram on viscosity versus storage time for three different solutions measured at 25° C.; and

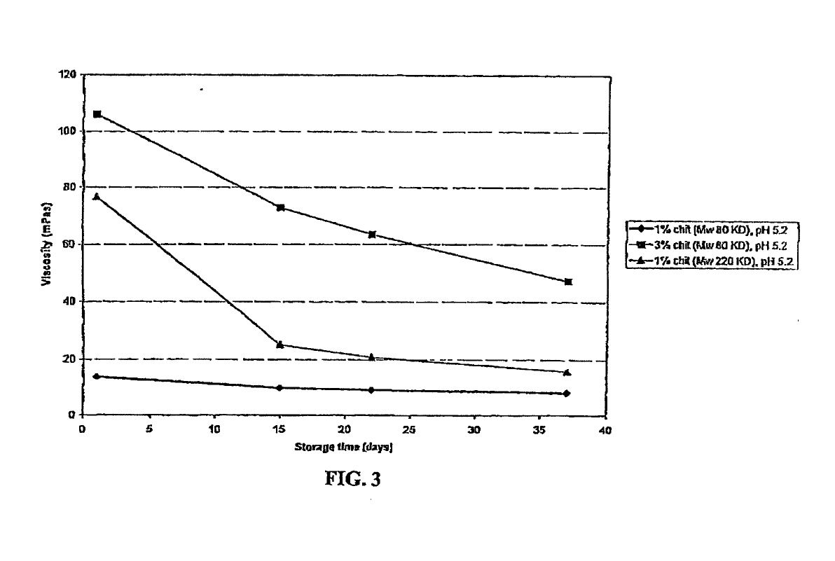

FIG. 3 is a similar diagram on viscosity versus storage time obtained by accelerated test at 40° C.

In the examples to follow, parts and percentages refer to weight unless otherwise indicated. Furthermore, the viscosity values discussed in this disclosure, including the claims, refer to values of newly prepared solutions, by which is understood solutions prepared no earlier than 24 hours before viscosity measurement. It is furthermore understood that solutions whose viscosity is modified in any way after mixing of the solution, e g through autoclaving, are not considered newly prepared until after such viscosity-modifying treatment.

The chitosan used in the following examples, except for Examples 3, 8, 13 and 18, has a molecular weight distribution as shown in FIG. 1 of the appended drawings. The viscosity is measured with Brookfield LVDV II+, UL Adapter at 25° C.

### EXAMPLE 1

Preperation of 100 ml Solution of 1%

5,8 g 87% glycerol is added to 95 ml of water. 0.3 ml acetic acid (99.9%) is added to the glycerol solution, and stirring is performed until a homogeneous solution is obtained. 1.0 g chitosan (base form, no acid addition salt thereof, \overline{Mw} about 80 kD, deacetylation degree 94% (Primex, Norway)) is added to the solution prepared, and stirring is maintained until all chitosan has been dissolved. The pH of this solution is about 5.2.

### EXAMPLE 2

Example 1 is repeated but using 3.0 g chitosan to form a 3%, solution. The amount acetic acid used is correspondingly increased to 0.9 ml.

### EXAMPLE 3

Example 1 is repeated but using 1.0 g chitosan having an \overline{Mw} of 220 kD (deacetylation degree 88%).

The three solutions prepared according to the examples above are subjected to storage at 25° C. and 40° C. FIG. 2 shows comparison between the three different solutions in a diagram showing viscosity versus storage time. The storage in this case is made at 25° C.

The diagram shown in FIG. 2 illustrates clearly the significantly improved stability that is obtained with the solution according to Example 1. The formulation according to Example 1 also results in a viscosity lying within the preferred range and enabling easy handling in connection with the application to the teats.

At a concentration of chitosan of 3% the viscosity is much higher and the stability of the viscosity of the solution is unacceptably variable with time.

On the other hand, using the solution according to Example 3 it can be seen that the viscosity value is subject to drastic decrease during is days making the solution according to Example 3 unsuited for practical use.

FIG. 3 shows the same viscosity data measured as an accelerated test at 40° C. The viscosity change is in this case even more pronounced for the solutions according to Examples 2 and 3.

### EXAMPLE 4

Example 1 is repeated but using glycolic acid instead of acetic acid in an equimolar amount. Similar improvement in viscosity stability at storage is obtained. Additionally, this acid has an advantageous skin conditioning effect.

### EXAMPLE 5

Example 1 is repeated but using a corresponding amount of the acetic acid addition salt of chitosan to form a 1% solution. Thus, no extra acetic acid is used in this case. Similar results are obtained.

### EXAMPLES 6-10

Examples 1-5 are repeated, but 0.02 g heparin is added to water before adding of glycerol, acetic acid and chitosan. Similar results with regard to viscosity stability are obtained.

### EXAMPLE 11-15

Examples 6-10 are repeated using dextran sulphate in an equimolar amount instead of heparin, Similar results with regard to viscosity stability are obtained.

### EXAMPLE 16-20

Examples 6-10 are repeated but using an equimolar amount of heparan sulphate instead of heparin. In viscosity stability storage tests similar results are obtained.

It is to be understood that the present invention as described is not restricted to the specific examples given above but as a scope only limited in accordance with the appended claims.

1. An aqueous solution for prophylactic treatment of teats of lactating mammals, comprising as a first component at least partially deacetylated chitosan, or an acid addition salt thereof, in a concentration of up to about 2% by weight of chitosan, the solution having a pH of from about 4 to about 6.8, and said first component having a molecular weight such that the viscosity of the solution is less than about 50 mPas.

2. An aqueous solution according to claim 1, further comprising a second component selected from heparin, heparan sulphate, and dextran sulphate, the weight ratio between said first and second components being from about 10:1 to about 100:1.

3. An aqueous solution according to claim 1 or 2, wherein said first component has a degree of deacetylation of no less than about 50%.

4. An aqueous solution according to claim 3, wherein the degree of deacetylation is no less than about 80%.

5. An aqueous solution according to claim 4, wherein the degree of deacetylation is in the range from about 85% to about 95%.

6. An aqueous solution according to any preceding claim, wherein the pH of the solution is maintained by the use of a mineral acid or organic acid.

7. An aqueous solution according to claim 6, wherein said acid is a weak organic acid.

8. An aqueous solution according to claim 7, wherein said weak organic acid is selected from acetic and glycolic acids.

9. An aqueous solution according to claim 6, wherein said first component is chitosan in base form at a concentration of from about 0.2 to about 1.5% by weight, the weight ratio between said first and second components is from about 40:1 to about 60:1, and said acid is selected from acetic and glycolic acids.

10. An aqueous solution according to claim 9, wherein the viscosity of the solution is less than about 25 mpas.

11. An aqueous solution according to claim 10, wherein the viscosity of the solution is from about 5 to about 20 mPas.

12. An aqueous solution according to claim 9, 10 or 11, wherein said molecular weight ia such that from about 70 to about 85% of the chitosan has a molecular weight of front about 20 to about 150 kD.

13. An aqueous solution according to claim 12, wherein the viscosity of the solution is such that about 80% of the chitosan has a molecular weight of from about 20 to about 150 kD.

14. An aqueous solution according to any one of claims 9 to 13, wherein said pH is from about 4.5 to about 6.

15. A method of prophylactic treatment of teats of lactating mammals to prevent onset of mastitis, comprising applying an aqueous solution according to any preceding claim to said teats in art anti-microbially active amount.

16. A method according to claim 15, wherein the application of said aqueous solution takes place in connection with milking operations.

17. A method according to claim 16, wherein said application is performed twice or thrice a day after milking.

18. A method according to claim 16, wherein said application is performed before milking.

19. A method according to any one of claims 15 to 18, wherein the application of said solution is performed by dipping or spraying so as to cover at last the tip of the teats.
